(19) United States
(12) Patent Application Publication
(10) Pub. No.: US 2003/0225318 A1
(43) Pub. Date: Dec. 4, 2003

(54) Intraocular pressure sensor

(76) Inventors: Valentino Montegrande, Coto De Caza, CA (US); Kevin S. Montegrande, San Francisco, CA (US)

Correspondence Address: LAW OFFICES OF ERIC KARICH, 2807 ST. MARK DR., MANSFIELD, TX, 76063, US

(21) Appl. No.: 10/452,109

(22) Filed: Jun. 2, 2003

Related U.S. Application Data
(60) Provisional application No. 60/384,632, filed on May 31, 2002.

(51) Int. Cl. A61B 5/00

(52) U.S. Cl. 600/300

(57) ABSTRACT

An intraocular pressure sensor for sensing pressure within an eye has a sensor reed and a strain gauge for sensing the pressure within they eye and for generating a sensor signal representative of the pressure. The intraocular pressure sensor also includes a transponder responsive to the strain gauge for converting the sensor signal to a pressure signal representative of the pressure. The transponder includes a processor, a sensor antenna adapted for receiving an interrogation signal generated from outside the eye, a modulator for converting the pressure signal into a response signal, and a power converter coupled with the sensor antenna for converting the interrogation signal to a power signal for energizing the processor. In addition to receiving the interrogation signal, the sensor antenna further functions to transmit the response signal out of the eye.

### CROSS-REFERENCE TO RELATED APPLICATIONS

This application for a utility patent claims the benefit of U.S. Provisional Application No. 60/384,632, filed May 31, 2002, which is hereby incorporated by reference in its entirety.

### STATEMENT REGARDING FEDERALLY SPONSORED RESEARCH

Not Applicable

### BACKGROUND OF THE INVENTION

1. Field of the Invention

The present invention relates to medical devices for monitoring conditions in an eye of a patient, and more particularly to an intraocular pressure sensor adapted to be positioned on or within the eye for measuring the intraocular pressure thereof.

2. Description of the Prior Art

Implantable devices for monitoring internal physiological conditions of a patient are known in the art. One such prior art device includes an implantable pressure transducer that transmits pressure signals out of the patient by means of a wire passing through the patient's skull. These types of devices are generally unsatisfactory due to increased risk of infection and patient discomfort caused by the externally extending wire.

Monitoring devices that are completely implantable within a patient are also known in the art. One such prior art device includes a sensor for sensing a physiological condition of the patient and a transmitter and battery assembly for transmitting the sensor signals out of the patient's body. These types of devices are also unsatisfactory for many types of medical conditions since the batteries are bulky and must be periodically replaced, thus necessitating additional surgery.

The state of the art includes the following:

Tremblay et al., U.S. Pat. No. 5,704,352, teaches an implantable, passive bio-sensor for monitoring internal physiological conditions of a patient. The bio-sensor includes at least one sensor or transducer for monitoring a physiological condition of the patient and a passive transponder that receives sensor signals from the sensor or sensors, digitizes the sensor signals, and transmits the digitized signals out of the patient's body when subjected to an externally generated interrogation signal. In one embodiment, the bio-sensor is incorporated into the sidewall of a shunt used for treating hydrocephalus for non-invasively monitoring the operation of the shunt.

Frenkel, U.S. Pat. No. 5,005,577, teaches an apparatus for monitoring intraocular pressure. The apparatus includes an implantable intraocular lens and at least one sensor apparatus responsive to intraocular pressure being affixed to the lens.

Jeffries et al., U.S. Pat. No. 6,193,656 B1, teaches an apparatus for monitoring intraocular pressure in an eye. The apparatus includes a miniature pressure sensor having an attachment for connecting the miniature pressure sensor to the iris of the eye or an intraocular lens. The miniature pressure sensor is preferably a Polysilicon Resonant Transducer (PRT).

Waters, Jr. et al., U.S. Pat. No. 4,922,913, teaches an intraocular pressure sensor that utilizes a small sensitive piezo-resistance strain gauge cell mounted in a curved semi-rigid holder which serves to position the planar pressure sensitive surface of the strain gauge cell in contact with the eyeball surface. Deformation of the strain gauge cell due to contact with the eyeball produces an output signal corresponding to the intraocular pressure. The sensor is small and can be worn in the eye like a contact lens for extended periods of time permitting the intraocular pressures to be accurately monitored under normal living conditions, including during sleep. Fine wires are led from the sensor out over the eyelid for connection to an external recording/monitoring apparatus.

The above-described references are hereby incorporated by reference in full.

The prior art teaches various sensors for monitoring physiological conditions within the body. However, the prior art does not teach an intraocular pressure sensor having the construction and benefits described herein. The present invention fulfills these needs and provides further related advantages as described in the following summary.

### SUMMARY OF THE INVENTION

The present invention teaches certain benefits in construction and use which give rise to the objectives described below.

The present invention provides an intraocular pressure sensor that includes a pressure sensor means for sensing the pressure within they eye, and a transponder means for generating a sensor signal representative of the pressure. The transponder means includes a processor means responsive to the sensor means for converting the sensor signal to a pressure signal representative of the pressure. The transponder means also includes a sensor antenna adapted for receiving an interrogation signal generated from outside the eye, a modulator for converting the pressure signal into a response signal, and a power converter coupled with the sensor antenna for converting the interrogation signal to a power signal for energizing the processor means.

In view of the foregoing, it is an object of the present invention to provide a sensor for placement or implantation on or within the eye of the patient for measuring the intraocular pressure within the eye.

It is another object of the present invention to provide a sensor that requires no batteries or other similar internal sources of power.

It is another object of the present invention to provide a biosensor that does not require a physical connection, by wire or otherwise, to an external source.

It is another object of the present invention to provide a biosensor that permits non-invasive queries of conditions inside the eye of the patient.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is an exploded perspective view of one embodiment of an intraocular pressure sensor of the present invention;

FIG. 2 is a block diagram of the general structure of the intraocular pressure sensor;

FIG. 3 is a block diagram of one particular embodiment thereof;

FIG. 4 is a side elevational view of a contact lens upon which the intraocular pressure sensor is operatively installed, illustrating how the intraocular pressure sensor can be positioned against an eye in one embodiment of the invention;

FIG. 5 is a perspective view of an intraocular lens upon which the intraocular pressure sensor has been operatively installed;

FIG. 6 is a side elevational view of a glaucoma drainage device upon which the intraocular pressure sensor has been operatively installed, the glaucoma drainage device being operatively installed in the eye;

FIG. 7 is a sectional view of the glaucoma drainage device illustrating the placement of the intraocular pressure sensor on a lumened tube of the glaucoma drainage device;

FIG. 8 is a block diagram of an activator/assessor device that is used in conjunction with the intraocular pressure sensor;

FIG. 9 is a perspective view of the activator/assessor device being used to transmit a query signal to the intraocular pressure sensor and receive a response signal in return;

FIG. 10 is a block diagram illustrating the activator/assessor device being used to query the intraocular pressure sensor for the purposes of calibration;

FIG. 11 is a block diagram illustrating the activator/assessor device being used to query the intraocular pressure sensor for purposes of ascertaining the pressure within the eye; and

FIG. 12 is a block diagram illustrating how the activator/assessor device is adapted to work through a wireless network with a central monitoring station.

### DETAILED DESCRIPTION OF THE INVENTION

The above-described drawing figures illustrate the invention, an intraocular pressure sensor 10 for sensing pressure in a system such as an eye 12 of an animal. The intraocular pressure sensor 10 may be used as part of an intraocular pressure sensor system 110, described in greater detail below.

### INTRAOCULAR PRESSURE SENSOR

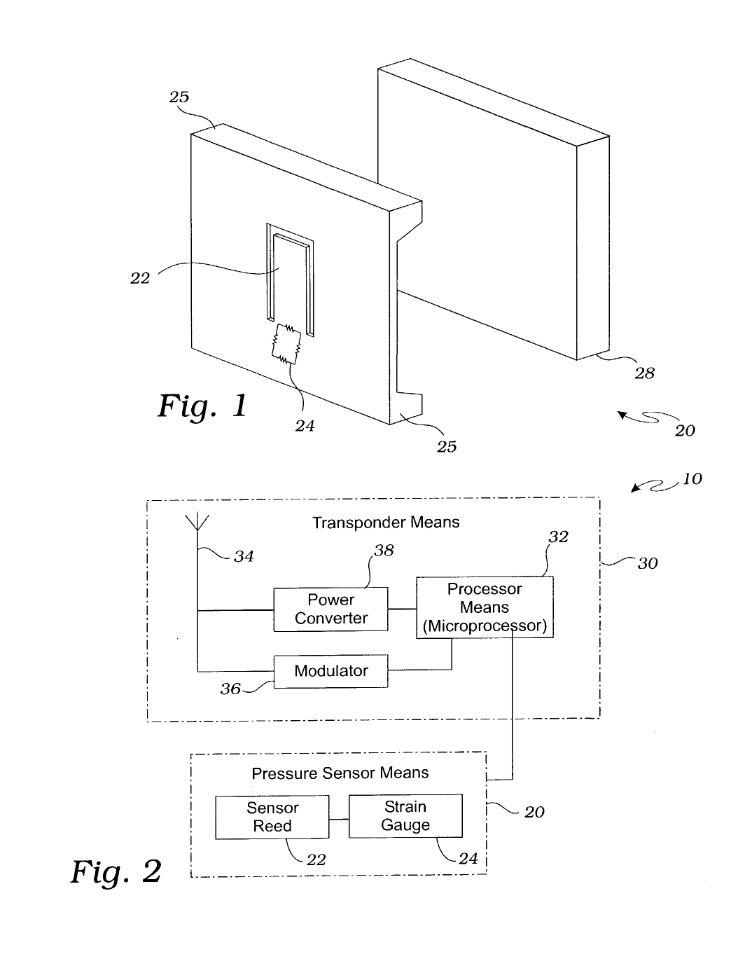

As shown in FIG. 1, the intraocular pressure sensor 10 is manufactured using microelectromechanical systems (MEMS) manufacturing techniques, so it is small enough to be readily adapted to many methods of continuously monitoring the pressure within the eye 12. The intraocular pressure sensor 10 may be positioned directly against the eye 12, implanted into the eye 12, or integrated with a medical device that is used in conjunction with monitoring or treating the eye 12. Several possible embodiments are described in greater detail below.

As shown in FIG. 2, the intraocular pressure sensor 10 includes a pressure sensor means 20 for sensing pressure within they eye 12 and for generating a sensor signal representative of the pressure; and a transponder means 30 electrically coupled with the pressure sensor means 20 for both powering the pressure sensing means 20 and reporting via wireless communication the pressure being sensed by the pressure sensor means 20.

In one embodiment, as shown in FIG. 1, the pressure sensor means 20 (shown in FIG. 2) includes a sensor reed 22 and a strain gauge 24. The sensor reed 22 is micro-machined, etched, or otherwise formed from a silicon chip body 25. The sensor reed 22 may include any arm, lever, or similar projection which may be moved, biased, or otherwise altered in configuration in response to changes of pressure within the eye 12. The sensor reed 22 is preferably a lever that is formed to be parallel to the surface of the silicon chip body 25.

The strain gauge 24 is operably positioned to measure the flexion of the sensor reed 22, either on the sensor reed 22 itself, or adjacent to the sensor reed 22 on the silicon chip body 25. For purposes of this application, the term strain gauge 24 shall include any form of strain gauge, including but not limited to a single Wheatstone bridge, a plurality of Wheatstone bridges, or any other form of circuitry with an equivalent operative sensor capability, in any configuration or arrangement.

As shown in FIG. 2, the transponder means 30 includes a processor means 32 responsive to the pressure sensor means 20 for converting the sensor signal to a pressure signal representative of the pressure, and a sensor antenna 34 adapted for receiving an interrogation signal 14 generated from outside the eye 12. In one embodiment, the processor means 32 is a microprocessor. In another embodiment, the processor means 32 includes a modulator 36 for converting the pressure signal into a response signal 16, and a power converter 38 coupled with the sensor antenna 34 for converting the interrogation signal 14 to a power signal for energizing the processor means 32. In addition to receiving the interrogation signal 14, the sensor antenna 34 further functions to transmit the response signal 16 out of the eye 12.

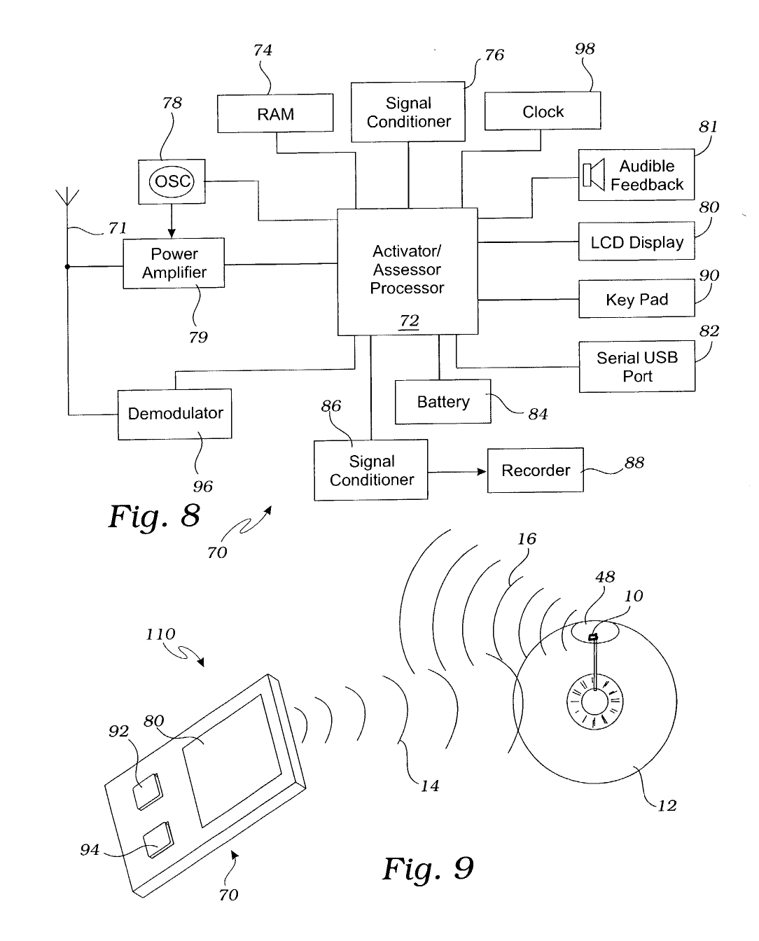

The sensor antenna 34 is electromagnetically coupled with an activator/assessor antenna 71 (shown in FIGS. 8 and 9) for receiving an interrogation signal 14, as described below. The power converter 38 is coupled with the sensor antenna 34 for extracting energy from the electromagnetic couple with the activator/assessor antenna 71. The power converter 38 converts this electromagnetic energy to a current signal for powering the processor means 32. The modulator 36 is coupled with the processor means 32 and the power converter 38 for receiving the digitized data from the processor means 32 and for modulating the interrogation signal 14 in accordance with the digitized data stream to alter the electronic characteristics of the interrogation signal 14 to generate a response signal 16 which can be detected by the activator/assessor device 70. The response signal 16 functions to transmit the pressure readings reported by the strain gauge 24. The modulation technique may include load-shift keying, or similar or equivalent techniques that may be devised by those skilled in the art.

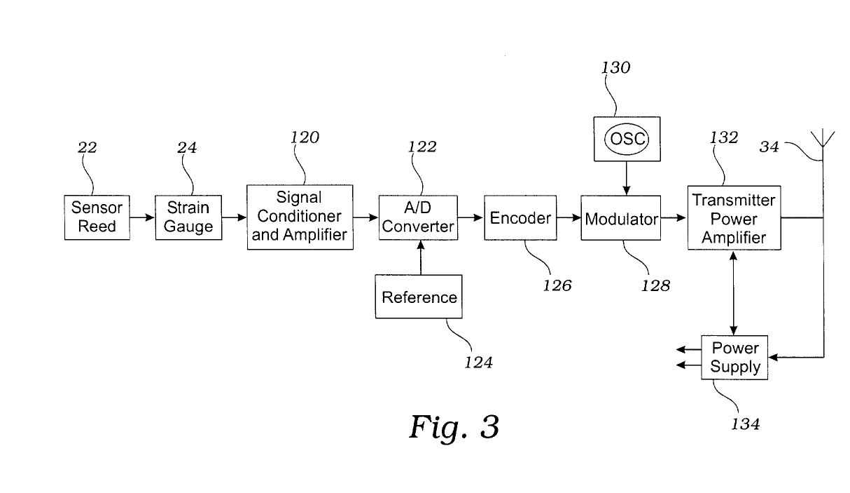

In one embodiment, the processor means 32 is a microprocessor. In another embodiment, as shown in FIG. 3, the processor means 32 includes a signal conditioner and amplifier 120, an A/D converter 122, a reference 124, an encoder 126, a modulator 128, a transmitter power amplifier 132, and an sensor oscilloscope 130. The signal conditioner and amplifier 120 is operably connected to the strain gauge 24 and to the A/D converter 122 (which is operably connected to the reference). The A/D converter 122 is also operably attached to the encoder 126, which is operably attached to the modulator 128. The sensor oscilloscope 130 is operably connected to the modulator 128 for sending the signal to the sensor antenna 34 through the transmitter power amplifier 132. The various elements are powered by the power supply 134, which receives its power from the sensor antenna 34.

In one embodiment, as shown in FIG. 1, the sensor reed 22 is integral with a silicon chip body 25 and etched therefrom using etching techniques known in the art. The silicon chip body 25 may be bonded to a wireless IC broadcast chip 28 that includes the various circuits described above. In an alternative embodiment, the various components could be formed on a single, or multiple chips, depending upon the specific requirements of the intraocular pressure sensor 10. In this form, the intraocular pressure sensor 10 is adapted to be positioned adjacent to, within, or otherwise operably engaged with the eye 12 so that the sensor reed 22 is operatively responsive to the pressure in the eye 12.

### ACTIVATOR/ASSESSOR DEVICE

As shown in FIGS. 8-11, intraocular pressure sensor 10 is preferably used as part of an intraocular pressure sensor system 110 that also includes an activator/assessor device 70. The activator/assessor device 70 functions to simultaneously energize the transponder means 30 and the pressure sensor means 20, and also receive and report the response signal 16.

In one embodiment, as shown in FIG. 8, the activator/assessor device 70 may include an activator/assessor processor 72 operably attached to RAM 74, Flash RAM 76, and a clock 98 for running the various software programs required to utilize the activator/assessor device 70. The activator/assessor device 70 may include a second oscilloscope 78 and a power amplifier 79 for transmitting through an activator/assessor antenna 71, and a demodulator 96 for receiving transmissions.

The activator/assessor processor 72 may also be operably attached to an LCD display 80, a serial USB port 82 or similar connection, a battery 84 or other power source, and various other elements that together enable the function if the activator/assessor device 70. The activator/assessor processor 72 is also operably attached to a signal conditioner 86 that is operably connected to a recorder 88 or equivalent means for recording the results of the signals received. The results can be stored in the RAM 74 or other memory means and later transmitted, downloaded, printed, or otherwise outputted to the doctor or other person tending to the treatment of the eye 12. For reporting data locally, the activator/assessor device 70 may include an LCD display 80 and audible feedback 81 such as speakers.

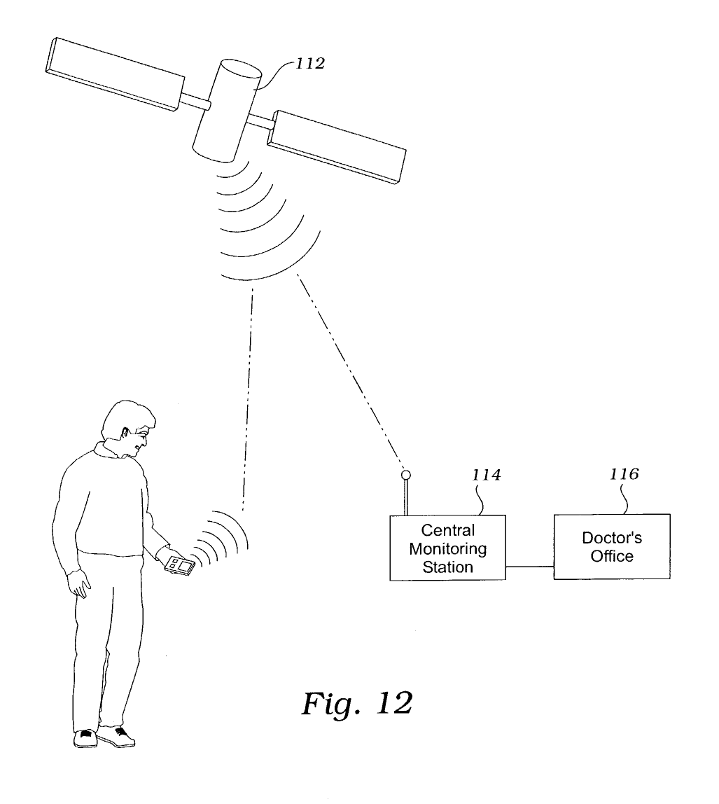

While the form of the activator/assessor device 70 can vary is size and shape depending upon the needs of the user, it is anticipated that the preferred embodiment will be a small handheld and battery 84 powered device, as shown in FIG. 9. In the embodiment illustrated, a keypad 90 is used to operatively control the activator/assessor device 70, although any control mechanisms known in the art could also be used. The keypad 90 includes a power button 92 and a manual actuation button 94; however, the keypad 90 could include a more complicated alphanumeric keyboard, voice actuation, or other control mechanism if desired. The power button 92 is used to power up the device, or turn it off to conserve battery 84 power. The manual actuation button 94 is used to trigger a query; however, it is also contemplated that the activator/assessor device 70 could also be programmed to automatically query the intraocular pressure sensor 10 at regular intervals as prescribed by a doctor, or upon receipt of a command signal from a central monitoring station (shown in FIG. 12, and described below).

In one embodiment, the activator/assessor processor 72 converts the analog signals from the sensors to digital signals and formats the digitized signals as a binary data stream for transmission out of the patient. The activator/assessor processor 72 is also operable for coding and formatting a unique device ID number (not shown) for transmission with the digitized transducer signals for use in identifying the device. In some embodiments of the invention, the activator/assessor processor 72 may be programmed for analyzing the signals before transmitting the signals out of the patient's body. For example, if the intraocular pressure sensor 10 is provided with a pressure transducer, the activator/assessor processor 72 can be programmed to alert the patient with an audible feedback in the event that the data is unusual and should be immediately reviewed by the doctor.

While the various features of the invention have been described in terms of specific embodiments, it should be noted that the invention is not limited thereto, but should be construed to include equivalent embodiments that can be developed by those skilled in the art when provided the teachings of the present invention.

### CONTACT LENS

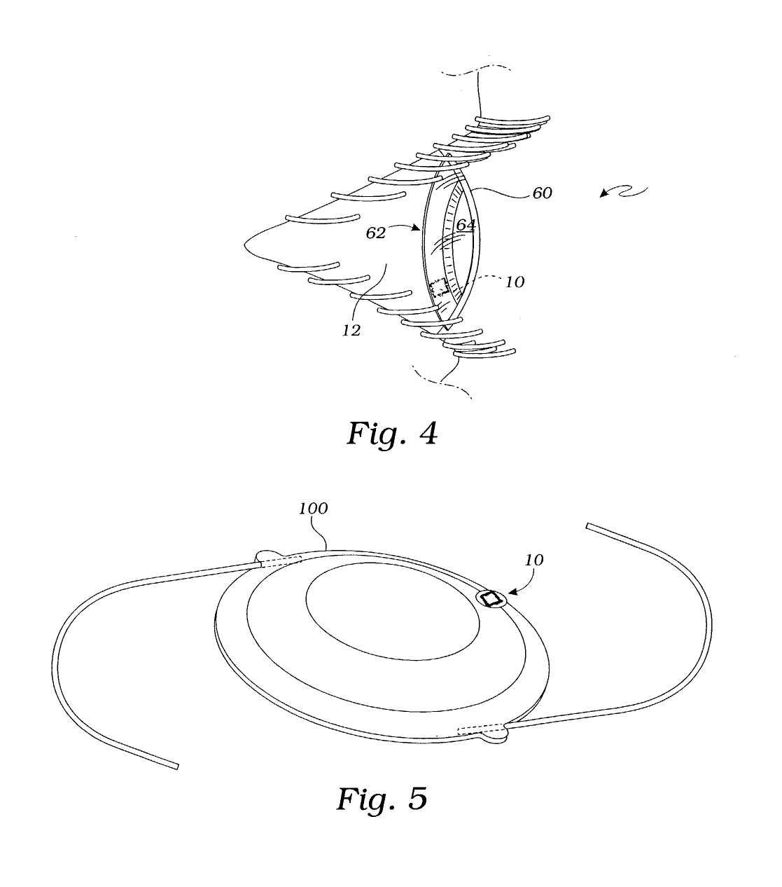

In a first embodiment, as shown in FIG. 4, the intraocular pressure sensor 10 may be adapted to be operably installed in a contact lens 60 or similar eye 12 canopy that is adapted to be placed directly on the eye 12. The intraocular pressure sensor 10 is used in conjunction with a contact lens 60 having an inner lens surface 62 and an opposing outer lens surface 64. The inner lens surface 62 is adapted to operably contact the eye 12. The intraocular pressure sensor 10 is operably mounted on the contact lens 60 so that the pressure sensor means 20 operably contacts the eye 12 when the contact lens 60 is operably placed on the eye 12.

### INTRAOCULAR LENS

In a second embodiment, as shown in FIG. 5, the intraocular pressure sensor 10 is adapted to be operably installed on an intraocular lens 100 that is adapted to be surgically implanted into the eye 12. The intraocular lens 100 may be constructed of polymethylmethacrylate (PMMA) and may be operatively installed in the eye 12 using surgical techniques well known in the art. The pressure sensor means 20 is operatively positioned on the intraocular lens 100 to enable measurement of the pressure of the eye 12.

### GLAUCOMA DRAINAGE DEVICE

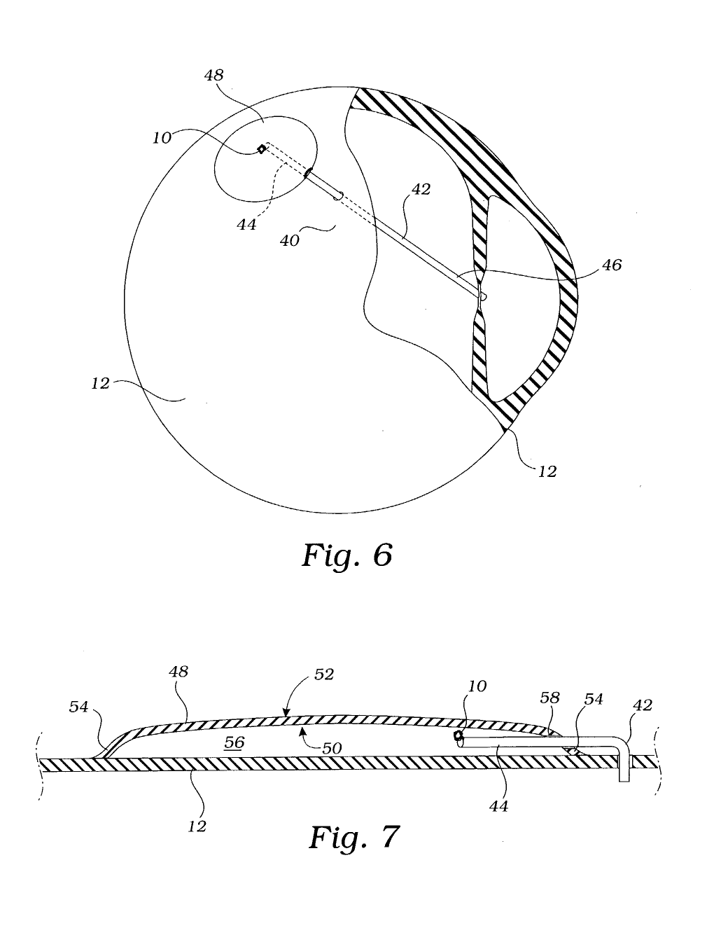

In a third embodiment, as shown in FIGS. 6-7, the intraocular pressure sensor 10 may also be adapted to be used on conjunction with a glaucoma drainage device 40. The glaucoma drainage device 40 includes a lumened tube 42 and an explant plate 48. The lumened tube 42 has a proximal end 44 and a distal end 46. The explant plate 48 has an internal surface 50 and an opposing external surface 52 that together terminate in a plate perimeter 54. The plate perimeter 54 is shaped to fit on the eye 12 and the internal surface 50 is concave to define an internal cavity 56 when the plate perimeter 54 is positioned on the eye 12. The proximal end 44 of the lumened tube 42 can be positioned through a tube aperture 58 of the explant plate 48 that is adjacent the plate perimeter 54. During surgery, the distal end 46 of the lumened tube 42 is positioned within the eye 12, to relieve pressure from within the eye 12 as directed by the doctor. The intraocular pressure sensor 10 is operable positionable adjacent the proximal end 44 for sensing flow pressure through the lumened tube 42.

### METHOD OF USE

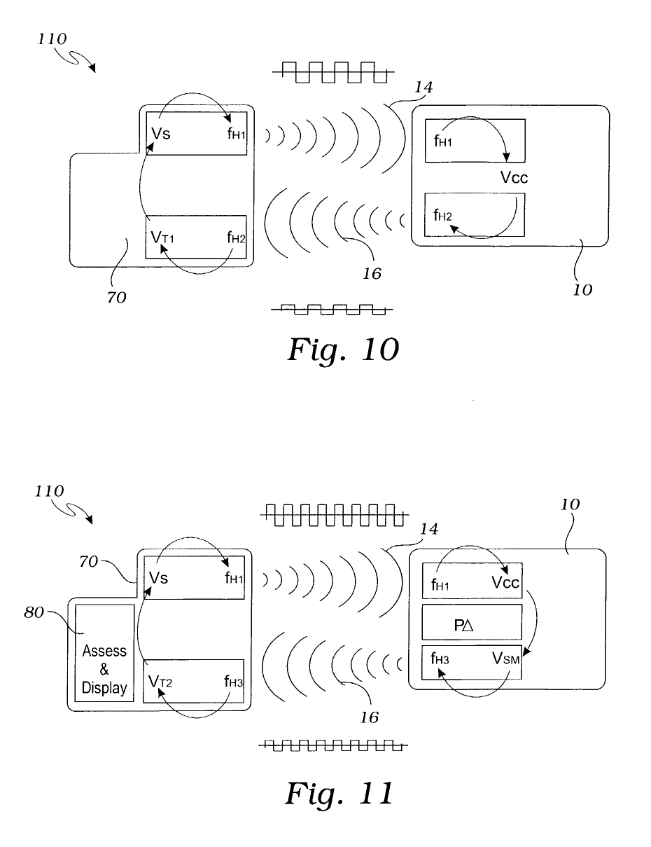

The intraocular pressure measurement system 110 may be used to measure the pressure an a system such as the eye, or other part of an animal such as a human, or any other system that may require continuous, remote pressure monitoring. As shown in FIG. 10, the intraocular pressure measurement system 110 is first calibrated. A seminal voltage Vs of the activator/assessor device 70 is used to generate a transmittal frequency FH1. The transmittal frequency FH1 is received by the intraocular pressure sensor 10 and used to generate a consistent core voltage Vcc, which in turn is used to generate a second transmittal frequency FH2. The second transmittal frequency FH2 is received by the activator/assessor device 70 and used to generate a terminal voltage Vt1.

As shown in FIG. 11, the intraocular pressure measurement system 110 may be used to measure the pressure sensed by the intraocular pressure sensor 10. The seminal voltage Vs is used to generate the transmittal frequency FH1, which is received by the intraocular pressure sensor 10 and used to generate the consistent core voltage Vcc. The consistent core voltage Vcc is then modified based upon the change in pressure measured by the intraocular pressure sensor 10, to a sensor-modified voltage Vsm. The sensor-modified voltage Vsm is used to generate a third transmittal frequency FH3. The third transmittal frequency FH3 is received by the activator/assessor device 70 and used to generate a second terminal voltage Vt2.

The difference between the terminal voltage Vt1 and the second terminal voltage Vt2 is representative of the pressure being measured by the intraocular pressure sensor 10. Those skilled in the art can devise many equivalent ways to practice this method, and such alternatives should be considered within the scope of the claimed invention.

### NETWORK

As shown in FIG. 12, the intraocular pressure measurement system 110 may be incorporated into a wireless network for reporting data regarding the pressure in the eye 12. The wireless network may include a receiver 112 such as a satellite system, a cellular transmitter/receiver, and/or any other commercial relay or system capable of handling network communications. Data from the activator/assessor device 70 is transmitted to the receiver 112 using any suitable protocol, such as 802.11 or other suitable network protocol. From the receiver 112, the data is then communicated to a central monitoring station 114 via a global computer network, a phone system, fiber optics, another wireless network, or any other network.

The central monitoring station 114 may process the data is many ways, including compiling and reporting the data, or simply forwarding the data to a doctor's office 116. The central monitoring station 114 and/or the doctor's office 116 may also actively monitor the data, alerting the user or the doctor to any spikes in pressure or other circumstances that may require medical care. For example, the central monitoring station 114 (or, of course, the doctor's office 116) may compile the data for later analysis by treating physicians, and store the data on the global computer network so that the user's physician may access the data at any time. If there is a dangerous and/or prolonged spike in intraocular pressure, the central monitoring station 114 may automatically page the treating physician and alert him or her to the situation, so that proper medical care may be immediately administered.

While the invention has been described with reference to at least one preferred embodiment, it is to be clearly understood by those skilled in the art that the invention is not limited thereto. Rather, the scope of the invention is to be interpreted only in conjunction with the appended claims.

What is claimed is:

1. An intraocular pressure sensor comprising:
a pressure sensor means for sensing pressure within they eye and for generating a sensor signal representative of the pressure;
a transponder means electrically coupled with the pressure sensor means, the transponder means including a processor means responsive to the sensor means for converting the sensor signal to a pressure signal representative of the pressure;
a sensor antenna adapted for receiving an interrogation signal generated from outside the eye;
a modulator for converting the pressure signal into a response signal; and
a power converter coupled with the sensor antenna for converting the interrogation signal to a power signal for energizing the processor means and transmitting the response signal out of the eye.

2. The intraocular pressure sensor of claim 1 wherein the pressure sensor means includes a strain gauge operatively positioned on a sensor reed.

3. The intraocular pressure sensor of claim 1 wherein the processor means is a microprocessor.

4. The intraocular pressure sensor of claim 1 wherein the processor means includes a signal conditioner and amplifier, an A/D converter, a reference, an encoder, a transmitter power amplifier, and an implant oscilloscope.

5. An intraocular pressure measurement system comprising:
an activator/assessor device having an activator/assessor processor and an activator/assessor antenna for transmitting an interrogation signal;
an intraocular pressure sensor having a pressure sensor means, and a transponder means electrically coupled with the pressure sensor means,
the pressure sensor means being adapted for sensing pressure within they eye,
the transponder means having a sensor antenna adapted for receiving the interrogation signal, a processor means responsive to the pressure sensor means for converting the interrogation signal into a pressure signal representative of the pressure,
a modulator for converting the pressure signal into a response signal, and
a power converter coupled with the sensor antenna for converting the interrogation signal to a power signal for energizing the processor means and transmitting the response signal out of the eye.

6. A method for measuring pressure in a system, the method comprising the steps of:
providing an intraocular pressure sensor and an activator/assessor device;
providing a seminal voltage in the activator/assessor device to generate a transmittal frequency;
receiving the transmittal frequency with the intraocular pressure sensor;
generating, in response to the transmittal frequency, a consistent core voltage;
generating a second transmittal frequency using the consistent core voltage;
receiving the second transmittal frequency with the activator/assessor device;
generating a terminal voltage from the second transmittal frequency;
measuring the terminal voltage;
positioning the intraocular pressure sensor in the system;
providing the seminal voltage of the activator/assessor device to generate the transmittal frequency;
receiving the transmittal frequency with the intraocular pressure sensor;
generating from the transmittal frequency the consistent core voltage;
modifying the consistent core voltage in proportion to the pressure adjacent the intraocular pressure sensor, to generate a sensor-modified voltage;
generating a third transmittal frequency using the sensor-modified voltage;
receiving the third transmittal frequency with the activator/assessor device;
generating a second terminal voltage from the third transmittal frequency; and
comparing the terminal voltage and the second terminal voltage to determine the pressure being sensed by the intraocular pressure sensor in the system.
